SEQUENCE LISTING (9 sequences)

SEQ ID NO: 1 - DNA, 5633 bp, synthetic construct
  source 1..5633: mol_type = other DNA
atgttaactaaacagcaaaatgaagacctgtctggacaagatgtaattgaaaaatatcctaaagagcagagatttactcttgctatactacaggatatacagagaaagtacaaatatataccaagagaagcactggagaatttagctaagtatttggacacgcctgtaagtagactgtatggtatggctactttttataaggcattaagccttactccaaagggggaaaacataataactgtatgtgatggaactgcttgccacgttgctggttctatggttgtaatggatgagcttgaaaaggcaataggaattagaccaggagaaactacagaagattttaaattttcaataaatacagttaactgtataggatgctgtgcaatagctcctgtcatgatgataaatgacaaatattttggaaatttaacacctaaactggttgaagaaattcttagtgagtataggagtgagagtgatgagtgataaaaaaactgtcaatatatgttgtgggacaggttgcttagctaaaggcagtatggaagtatatgaagaaatgaaggcacaaatagctaaattaggcgcaaatgcagaagtaagtgttaaattaaaagcaacaggttgtgatggattgtgtgaaaaaggtcctgtactgaaaatatatccagatgacattgcatattttaaagttaaagtagaagatgtagaagacgtagtaaaaaagacattgatgaatggggaaataattgaaaagttattatattttgaaactgctacaaaacagagattaagaaatcataaagaaagtgaattctgtaaaagacaatacaaaattgccctcaggaatgttggtgaaatagatccaataagtttggaagattacgttgaaagaggcggatataaagctcttagaaaagcaatagctaatatgaagcctgaagatgtacttggagaaatatcaaaatcaggtcttagaggaagaggtggagcaggattcccaacaggacgtaaatggaaaactgctgcagacattgatacagcacctatatatgtagtatgtaatggtgatgagggagaccctggagcatttatggatagaagtataatggagggagatcctaacagtgttatagaaggtatgacattgtgtgcttatgcagtaggaggtacaaatggatttgcttatataagagatgaatatggacttgctgtagaaaatatgcagaaagctattaataaagcaaaagaagaaaatttattaggtaataatatattgggaactgatttttccttcgatatacagatagtaagaggcggaggagcttttgtatgtggtgagtctactgcacttatgtcatctatagaaggtatggtaggagaacctagagctaaatatatacacactacagaaaaaggattatggggacaacctactgttttaaataatgtagaaacttgggccaatgtacctataataattgaaaaaggtggagattggtatcatgctataggaactatggagaagagcaagggaacaaaagtattctcactagttggaaaagttaagaatactggacttgtagaagtacctatgggaactactcttagagaaataatatatgatattggcggtggagtattaaatgacagaaagtttaaggcagttcaaataggtggaccttcaggaggatgtttaccgtctgaatatttagacttgccagtggattatgacactttggttaaagcagattctatgatgggttccggcggaatgatcgtaatggatgatagaacctgtatggtggatgtaactagatattacttgagtttcctagctgaagaatcttgtggaaagtgtgtaccttgtagagaaggcgtaaagaggatgcttgagatactcactgacatgtgcaatggtgatggaaaagaaggggacatagaagaacttcttgaaatatgttccatgacaagcaaggcatctctgtgcagccttggtaagagtgctccaaatccagtaaaagctgctataagatattttagagatgaatttgaagagcatataaagaataaaagatgtagagcaggagtttgtaagaaacttactacatttggtatagatcaagataaatgtattggatgcgatatgtgtaaaaagaattgtccggctgattgtataacaggggaaattaagaaaccacatacaatagatgcggataagtgcttgagatgcggtaactgcatgaacatctgtaagcttaatgctgttaaggttctatagggaggtgaatgtagatatgaaaattacaatagatggaaaagcttgtgaagctgaaaaaggagaattcatattacaaatagcaagaagaaataatatatatatacctacactgtgtcatagtgatgcattacctggacttgctagctgtaggctgtgtatagttaaagtaatagataggggacgtgcaaagatagtaacttcctgtatattccctgtaagtaaagaagtagaagttgtaactaatgacgatgaaataaatagaatgaggaaaaacatagttatgcttctaaaagtaagatgccctgaaaataaagaggtaaatgaattagctaaagcctttggagtagaggaaaagagagtaaagaggttcaaattggatccagaacaaaattgtgttttgtgcggactttgtgcaaaagcttgcaaggaattaggtactggagcaatttcaactgttaacaggggtatgtataaagaagtagcaactccatatcatgaatcttcaccagaatgtataggatgtgcttcttgtgctaatgtttgcccaactaatgcaataaaagttgtggataaagatggagaaagagaaatatggggcaaaaaatttaagatggttaaatgtgatttgtgcggagaatattttgctactgaagaacatgtaaagtatgcttatgatagattgggagtaaatcagccagaaaagcttatgtgcagcagctgcaagaagaaagttacagccaaagatgtcaaaagtatttttgagaacgtgtaaaaattaaaagaggggtggtaatttaaatgaaaccagagtttaattcttttgtaatagccgatcctgacaagtgcataggctgtagatcttgtgagattgcttgtgctgcaaaacatagagaagatactcaaggaaaaactattggaactatgaataataaagttactccaaggttattctttgttaaaaataaaggaaatgtaatgccagtacaatgcagacattgtgaggatgcaccatgtctaaatgcctgcccagttaatgctatagttgaaaaagatggaagtatcattataaatgaaagtgtatgtatagggtgtcagacctgtacaatagtatgtccggtaggtgctgtaagtttacttcctagaactcaaggtaaagtggttacaggaggaattcaggttaaagtaagagcagcagcttataaatgtgatttatgtaaggaagagggaggagaacctgcctgcgtcaaggaatgtccaaaagaagccttgaggttagtagatcctagagaagataaaaaagatcgtagtgtgaaagctgctatggaactgttaaatataaacgcaaatctctaatttataagttataaaaatccggaaaagaaaggaatgttaaatatgccaactagtacttctatgataaatatagatgaagaattatgtacaggctgcagacgatgtgcggatgtctgccctgtagatgctatagaaggtgaacagggtaaacctcagaagataaatactgaaaagtgtgttatgtgcggacaatgcattcaagtttgtaaaggctatcaatctgtatacgatgatgttcctactccagttagcaaaaggttatttgatagaggattgttaaaggaagtagatgaaccattatttgcagcatataataaaggtcaggcaaaaagagttaaagaaattttacaaaacaaagatgtatttaaaattgtgcaatgtgcacctgctgtaagagtcgctataggagaggattttggattacctcttggaactttaagcgagggaaaaatggcagctgcactcagaaaattaggatttgacaaagtatatgatacaaactttggtgcagatcttactataatggaagaaggtagtgagttactaaaaagagtagctgaaggtggagttttgccaatgtttacttcttgttgtccagcatgggtaaaatatgcagaacaaacatatccagaacttttacctcatctttcaagttgtaagtctccaaatcagatggctggagctatatttaaaacttatggagcggagataaataaagttaatccggctaaaatttataatgtatctgttatgccatgtacatgcaaggaatttgaaagtgaaagagaagaaatgcatgacagtggacacagggatgtagatgcagttataactacaagggaattagcacaactgttcaaagatgctaatatagattttaatactattgaagaaggacagtttgatactcctcttggtatgtatactggcgcaggaactatatttggtgctacaggtggagttatggaagcagcacttagaactggatatgaactttatactaaaaaaactattccaagtatagatcttactatggtaagaggtggagaaggttttagaactgctgaagtagatttaggagatattagactaaaagtaggaatagtttccggcttaaaaaatgtaaaagacgttatggaatcagtaaaggcaggtaaatgtgatttacactttatagaggttatgacctgtcctcaaggatgcataagtggtggaggacaacctaaggttatacttgattcagataaagaagaagcttataataataggaaaaagggactatataatcatgacgctaatcttacttatagaaaatcacatgaaaatccagaaataaagaaaatatatgatgagttcttagacaaaccattaggagctaagtctcatgaattattgcacactaaatatatctcaagaaaaaaggagagttaataaaatgaagaattgcctcgtagtagcggatcctaataaatgcataggatgtaggacttgtgaagcagcttgtggtattgcacattcagggggagatttttttaatacaaatgtatccaaaattaattttaatcctcgcttaaatgtgataaaaactgctaaagtaagtgctcctgttcaatgcagacaatgcgaagatgcaccttgcggcaaagcttgcccagttaacgctatttcaaatgaaaatggttatgttagtgtagataaagatgtatgtgttggatgtaaaatctgcatgttagcttgtccttttggagctattgaattggcttctcaatataaggacggaaaaattgtagatcaaaagggacttaagatgagtgaagaaggtaatcctactgtaaatggaaaaggaagagtggtagcaaataagtgtgatctctgtcaggatagggatggaggacctgcttgcatagaagtttgtcctacaaaatctctcaaattagttacttatgacgacaataataatatagttgaaaaaaaagatgacgacgaacgtgaagtaagctaa

SEQ ID NO: 2 - DNA, 187 bp, synthetic construct
  source 1..187: mol_type = other DNA
taatttctgctaatacatttttaaataaggtaattaaaataattttaaattaaatatgttatatgttttaaaattatttcttaagcatagaggctcaaatctttgatttagagctaatatcttattccttctaatattttaagggggaaatcaattataatattcaaatgggagggtgaagtattta

SEQ ID NO: 3 - DNA, 295 bp, synthetic construct
  source 1..295: mol_type = other DNA
taaatacttcaccctcccatttgaatattataattgatttcccccttaaaatattagaaggaataagatattagctctaaaccaaagatttgagcctctatacttaagaaataattttaaaacatataacacatttaatttaaaattattttaattaccttatttaaaaattaaaatataaacttagtcctatttaattagggactaattattaacattaaatattatattatttttatataactatatgattaattttttaagattatattcaaaaatatgttaacagaaatta

SEQ ID NO: 4 - protein, 161 aa, synthetic construct
MLTKQQNEDLSGQDVIEKYPKEQRFTLAILQDIQRKYKYIPREALENLAKYLDTPVSRLYGMATFYKALSLTPKGENIITVCDGTACHVAGSMVVMDELEKAIGIRPGETTEDFKFSINTVNCIGCCAIAPVMMINDKYFGNLTPKLVEEILSEYRSESDE

SEQ ID NO: 5 - protein, 599 aa, synthetic construct
MSDKKTVNICCGTGCLAKGSMEVYEEMKAQIAKLGANAEVSVKLKATGCDGLCEKGPVLKIYPDDIAYFKVKVEDVEDVVKKTLMNGEIIEKLLYFETATKQRLRNHKESEFCKRQYKIALRNVGEIDPISLEDYVERGGYKALRKAIANMKPEDVLGEISKSGLRGRGGAGFPTGRKWKTAADIDTAPIYVVCNGDEGDPGAFMDRSIMEGDPNSVIEGMTLCAYAVGGTNGFAYIRDEYGLAVENMQKAINKAKEENLLGNNILGTDFSFDIQIVRGGGAFVCGESTALMSSIEGMVGEPRAKYIHTTEKGLWGQPTVLNNVETWANVPIIIEKGGDWYHAIGTMEKSKGTKVFSLVGKVKNTGLVEVPMGTTLREIIYDIGGGVLNDRKFKAVQIGGPSGGCLPSEYLDLPVDYDTLVKADSMMGSGGMIVMDDRTCMVDVTRYYLSFLAEESCGKCVPCREGVKRMLEILTDMCNGDGKEGDIEELLEICSMTSKASLCSLGKSAPNPVKAAIRYFRDEFEEHIKNKRCRAGVCKKLTTFGIDQDKCIGCDMCKKNCPADCITGEIKKPHTIDADKCLRCGNCMNICKLNAVKVL

SEQ ID NO: 6 - protein, 263 aa, synthetic construct
MLLRFYREVNVDMKITIDGKACEAEKGEFILQIARRNNIYIPTLCHSDALPGLASCRLCIVKVIDRGRAKIVTSCIFPVSKEVEVVTNDDEINRMRKNIVMLLKVRCPENKEVNELAKAFGVEEKRVKRFKLDPEQNCVLCGLCAKACKELGTGAISTVNRGMYKEVATPYHESSPECIGCASCANVCPTNAIKVVDKDGEREIWGKKFKMVKCDLCGEYFATEEHVKYAYDRLGVNQPEKLMCSSCKKKVTAKDVKSIFENV

SEQ ID NO: 7 - protein, 184 aa, synthetic construct
MKPEFNSFVIADPDKCIGCRSCEIACAAKHREDTQGKTIGTMNNKVTPRLFFVKNKGNVMPVQCRHCEDAPCLNACPVNAIVEKDGSIIINESVCIGCQTCTIVCPVGAVSLLPRTQGKVVTGGIQVKVRAAAYKCDLCKEEGGEPACVKECPKEALRLVDPREDKKDRSVKAAMELLNINANL

SEQ ID NO: 8 - protein, 465 aa, synthetic construct
MLNMPTSTSMINIDEELCTGCRRCADVCPVDAIEGEQGKPQKINTEKCVMCGQCIQVCKGYQSVYDDVPTPVSKRLFDRGLLKEVDEPLFAAYNKGQAKRVKEILQNKDVFKIVQCAPAVRVAIGEDFGLPLGTLSEGKMAAALRKLGFDKVYDTNFGADLTIMEEGSELLKRVAEGGVLPMFTSCCPAWVKYAEQTYPELLPHLSSCKSPNQMAGAIFKTYGAEINKVNPAKIYNVSVMPCTCKEFESEREEMHDSGHRDVDAVITTRELAQLFKDANIDFNTIEEGQFDTPLGMYTGAGTIFGATGGVMEAALRTGYELYTKKTIPSIDLTMVRGGEGFRTAEVDLGDIRLKVGIVSGLKNVKDVMESVKAGKCDLHFIEVMTCPQGCISGGGQPKVILDSDKEEAYNNRKKGLYNHDANLTYRKSHENPEIKKIYDEFLDKPLGAKSHELLHTKYISRKKES

SEQ ID NO: 9 - protein, 205 aa, synthetic construct
MNYCTLNISQEKRRVNKMKNCLVVADPNKCIGCRTCEAACGIAHSGGDFFNTNVSKINFNPRLNVIKTAKVSAPVQCRQCEDAPCGKACPVNAISNENGYVSVDKDVCVGCKICMLACPFGAIELASQYKDGKIVDQKGLKMSEEGNPTVNGKGRVVANKCDLCQDRDGGPACIEVCPTKSLKLVTYDDNNNIVEKKDDDEREVS

(19) United States
(12) Patent Application Publication
(10) Pub. No.: US 2025/0137016 A1
(43) Pub. Date: May 1, 2025

(54) METHODS FOR EFFICIENT FERMENTION BROTH RECYCLE

(71) Applicant: SYNATA BIO, INC., Warrenville, IL (US)

(72) Inventors: David Harvey KEATING, Warrenville, IL (US); Matthew Theodore KISSINGER, Warrenville, IL (US)

(73) Assignee: SYNATA BIO, INC., Warrenville, IL (US)

(21) Appl. No.: 18/728,395

(22) PCT Filed: Jan. 11, 2023
(86) PCT No.: PCT/US2023/060490

§ 371 Date: Jul. 11, 2024

Related U.S. Application Data
(60) Provisional application No. 63/298,426, filed on Jan. 11, 2022.

(51) Int. Cl. C12P 7/06; A23K 10/16; C05F 11/08; C12N 9/02; C12R 1/01

(52) U.S. Cl. CPC C12P 7/065; A23K 10/16; C05F 11/08; C12N 9/0067; C12Y 112/99006; C12R 2001/01

(57) ABSTRACT

Disclosed are methods for efficient fermentation broth recycle, methods for improving bottoms recycle, and methods for converting CO, CO₂, and optionally H₂ to ethanol and other oxygenated products, the methods comprising providing to a bioreactor a gaseous substrate comprising CO, CO₂, and optionally H₂, at least one acetogenic carboxydotrophic bacterium, and a liquid nutrient medium, and providing conditions within the bioreactor for the at least one acetogenic carboxydotrophic bacterium to convert CO, CO₂, and optionally H₂ to at least one oxygenated product. Also disclosed are methods for preparing animal feed and for preparing fertilizer.

### CROSS REFERENCE TO RELATED APPLICATIONS

This patent application claims the benefit of co-pending U.S. Provisional Patent Application No. 63/298,426, filed Jan. 11, 2022, which is incorporated by reference herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 16,539 Byte XML file named “766306.xml,” dated Jan. 6, 2023.

### BACKGROUND OF THE INVENTION

It is desirable to use microorganisms to convert gases containing carbon monoxide (CO), carbon dioxide (CO₂), and/or hydrogen (H₂), such as industrial waste gas or syngas, into a variety of products, such as fuels and chemicals using fermentation. The low ethanol tolerance of organisms that can consume CO, CO₂, and H₂ requires the use of continuous fermentation techniques, whereby broth containing ethanol, acetate, and cells are removed from the fermentor and new growth medium is added. The ethanol and cells are then recovered from the removed broth. The ethanol-depleted removed broth then needs to be subjected to wastewater treatment prior to release, which adds significantly to capital and process costs. For example, a commercial-scale bioreactor may contain in excess of 1 million liters of aqueous broth and creating a wastewater treatment system to handle all of the ethanol-depleted removed broth would require significant capital costs.

Accordingly, there remains a need for methods that reduce the requirement for expensive wastewater treatment and high water use associated with continuous fermentation. One approach to reducing this burden on wastewater treatment is to recycle the removed broth back to the fermentor (referred to as bottoms recycle). However, the percentage of removed broth that can be re-introduced to the fermentor is limited by the tolerance of the microorganism for this recycled broth. Therefore, improved microorganisms that permit more efficient wastewater recycling are advantageous.

### BRIEF SUMMARY OF THE INVENTION

An aspect of the invention provides a method for efficient fermentation broth recycle, the method comprising providing to a bioreactor a gaseous substrate comprising CO, CO₂, and optionally H₂, at least one acetogenic carboxydotrophic bacterium, and a liquid nutrient medium, and providing conditions within the bioreactor for the at least one acetogenic carboxydotrophic bacterium to convert CO, CO₂ and H₂ to at least one oxygenated product, wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

Another aspect of the invention provides a method for improving bottoms recycle, the method comprising providing to a bioreactor a gaseous substrate comprising CO, CO₂, and optionally H₂ at least one acetogenic carboxydotrophic bacterium, and a liquid nutrient medium, and providing conditions within the bioreactor for the at least one acetogenic carboxydotrophic bacterium to convert CO, CO₂, and optionally H₂ to at least one oxygenated product, wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

A further aspect of the invention provides a method for converting CO₂, CO and H₂ to ethanol, the method comprising providing to a bioreactor a gaseous substrate comprising CO, CO₂, and optionally H₂ at least one acetogenic carboxydotrophic bacterium, and a liquid nutrient medium, and providing conditions within the bioreactor for the at least one acetogenic carboxydotrophic bacterium to convert CO, CO₂ and H₂ to at least one oxygenated product, wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

Additional aspects include methods for preparing animal feed and for preparing fertilizer.

### BRIEF DESCRIPTION OF THE SEVERAL VIEWS OF THE DRAWING(S)

FIGS. 1A and 1B are images showing plots of the mean read density of a gene cluster in organisms SB1 (SB1 is a bacterium in an aspect of the present invention) (FIG. 1A) and Clostridium autoethanogemum (FIG. 1B) (as determined by RNA-seq analysis). The following genes are illustrated in FIGS. 1A and 1B: fdhH, moeA, moeB, fdhD, nuoE, nuoF, nuoG, hydN, and hydrogenase-1. Higher lines on the plots are proportional to high levels of gene expression.

FIGS. 2A and 2B are graphs showing the average amount of gene expression for hydrogenase-1 and formate dehydrogenase gene clusters in organisms SB1 (FIG. 2A) and C. autoethanogemim (FIG. 2B). Formate dehydrogenase represents the average gene expression of the genes associated with formate dehydrogenase (fdhH-fdhD) and hydrogenase-1 represents the average gene expression of the genes associated in hydrogenase (nuoE-hydN).

FIG. 3 is a graph depicting hydrogen uptake of C. autoethanogemim (C. auto) over time during fermentation in the presence of syngas. After 558 hours (arrow), the cell media was switched to 85% bottoms recycle of fermentation broth.

FIG. 4 is a graph depicting hydrogen uptake of SB1 over time during fermentation in the presence of syngas. At 143 hours (arrow), the cell media was switched to 75% bottoms recycle of fermentation broth, and at 307 hr, the percentage of bottoms recycle was increased to 89%.

FIG. 5 is a graph depicting acetate concentration in fermentation broth over time wherein C. autoethanogemim (C. auto) is in the presence of syngas as described in FIG. 3.

FIG. 6 is a graph depicting acetate concentration in fermentation broth over time wherein SB1 is in the presence of syngas as described in FIG. 4.

FIGS. 7A and 7B are bar graphs showing the averaged pan gene expression for the formate dehydrogenase and hydrogenase genes in SB1 (FIG. 7A) and C. autoethanogemum (FIG. 7B).

FIG. 8 is a bar graph showing ethanol and acetate volumetric productivity in SB1 and C. autoethanogemim.

FIG. 9 is a bar graph showing the rates of CO, hydrogen, and CO₂ uptake in SB1 and C. autoethanogemim.

FIG. 10 is a schematic of the Wood-Ljungdahl pathway.

FIG. 11A is a schematic of a plasmid in accordance with an aspect of the invention. pSyn63 contains the intergenic (I.G) region (SEQ ID NO: 3) from C. autoethanogenum and the initial 100 bp of nuoE cloned into plasmid pMTL83151. Plasmid pSyn64 contains the intergenic region from SB1 (SEQ ID NO: 2) and the first 100 bp of nuoE. The first 100 bp of nuoE is identical in both C. autoethanogenum and SB1.

FIG. 11B shows a plot of expression of the nuoE gene in pSyn63 and pSyn64 transformed into strain SB1. Plasmid pMT183151 was used as a negative control in that it did not contain the nuoE gene. Error bars represent standard deviation.

### DETAILED DESCRIPTION OF THE INVENTION

As noted above, one approach to reducing wastewater treatment costs and high water usage associated with continuous fermentation is to recycle the removed broth back to the reactor. Acetate is also produced during fermentation of CO, CO₂ and H₂ and, unlike ethanol, is not efficiently removed from the fermentor broth by methods such as distillation. Therefore, in the absence of conversion by the microorganism the acetate in the media will increase during fermentor broth recycling, which will lead to toxicity and reduced fermentation performance. Although the fermentation broth could be discarded in the event that the concentration of acetate becomes excessive, nutrients for the fermentation would also be lost. In addition, the acetate represents lost revenue, and an increased burden on wastewater treatment.

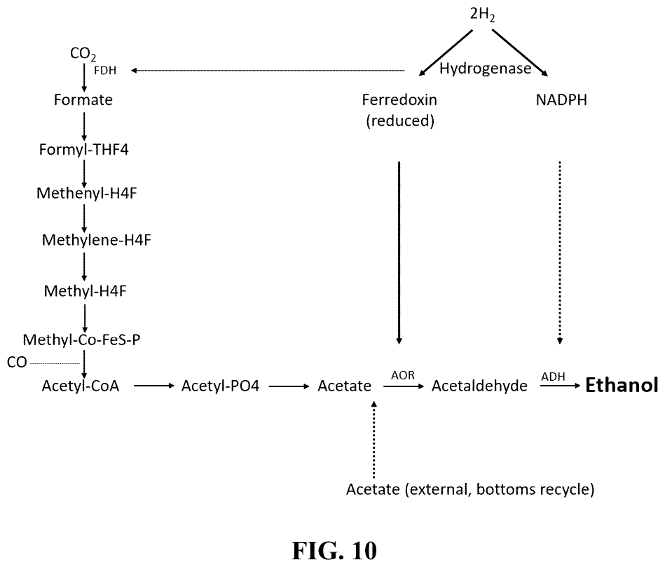

FIG. 10 illustrates the Wood-Ljungdahl pathway and primary bifurcating hydrogenase during production of ethanol from syngas. Hydrogenase fulfills two roles in production of ethanol. First, it provides reducing equivalents for the production of formate from CO₂ by formate dehydrogenase (FDH; denoted by arrow). Second, it produces reduced ferredoxin and NADPH that is used to convert acetate to acetaldehyde (via aldehyde oxidoreductase (AOR)) and its conversion to ethanol (via alcohol dehydrogenase (ADH)). The dotted line from NADPH to ADH is used to represent that ADH can use NADPH or NADH (which can be produced from NADPH). Therefore, hydrogen uptake is needed for efficient conversion of acetate to ethanol, and for efficient recycling of the removed broth. Acetate can be produced directly from the Wood-Ljungdahl pathway as shown, or can be present in recycled cell broth (bottoms recycle). THF4-refers to tetrahydrofolate.

It has been discovered that an increase in efficiency of fermentation broth recycle can be achieved by using an acetogenic carboxydotrophic bacterium comprising an upstream region sequence comprising at least about 90% identity to SEQ ID NO: 2. The upstream region sequence increases the expression of a hydrogenase gene cluster (e.g., a hydrogenase gene cluster comprising at least about 90% identity to SEQ ID NO: 1) and thereby allows the acetogenic carboxydotrophic bacteriumb to produce more hydrogenase enzyme and more hydrogenase enzyme would be expected to lead to elevated production of NAD(P)H and reduced ferrodoxin.

An aspect of the invention provides methods for efficient fermentation broth recycle, the methods comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂, and optionally H₂ to at least one oxygenated product, wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the hydrogenase gene cluster comprises at least about 90% identity to SEQ ID NO: 1, the upstream region sequence comprises at least about 90% identity to SEQ ID NO: 2, and the conditions within the bioreactor create a bioreactor broth.

An aspect of the invention provides methods for efficient fermentation broth recycle, the methods comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂, and optionally H₂ to at least one oxygenated product, wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

In an aspect, the oxygenated product is acetic acid (“acetic acid” and “acetate” are used interchangeably herein), butyrate, butanol, propanol. In an aspect, the product is ethanol.

In an aspect, the gaseous substrate comprising CO, CO₂, and optionally H₂ is a synthesis gas (syngas), such as syngas obtained by gasification of coal or refinery residues, gasification of biomass or lignocellulosic material, or reforming of natural gas. In another aspect, the syngas may be obtained from the gasification of municipal solid waste or industrial solid waste.

The gaseous substrate may comprise at least CO, such as about 1, about 2, about 5, about 10, about 20, about 30, about 40, about 50, about 60, about 70, about 80, about 90, or about 100 mol % CO. The gaseous substrate may comprise a range of CO, such as about 20 to about 80, about 30 to about 70, or about 40 to about 60 mol % CO. Preferably, the substrate comprises about 40 to about 70 mol % CO (e.g., steel mill or blast furnace gas), about 20 to about 30 mol % CO (e.g., basic oxygen furnace gas), or about 15 to about 45 mol % CO (e.g., syngas). In some aspects, the gaseous substrate may comprise a relatively low amount of CO, such as about 1 to about 10 or about 1 to about 20 mol % CO. In some aspects, the gaseous substrate comprises no or substantially no (<about 1 mol %) CO.

The gaseous substrate may optionally comprise H₂. For example, the gaseous substrate may comprise about 1, about 2, about 5, about 10, about 15, about 20, or about 30 mol % H₂. In some aspects, the gaseous substrate may comprise a relatively high amount of H₂, such as about 60, about 70, about 80, or about 90 mol % H₂. In another aspect, the gaseous substrate comprises no or substantially no (<about 1 mol %) H₂ (e.g., when derived from steel mill gas). The H₂ may be derived from or produced by any suitable process, including the formation of H₂ using electrodes.

The gaseous substrate may comprise CO₂. For example, the gaseous substrate may comprise from about 1 to about 80, from about 1 to about 70, from about 1 to about 60, from about 1 to about 50, from about 1 to about 40, from about 1 to about 30, from about 1 to about 25, from about 1 to about 20, from about 1 to about 15, from about 1 to about 10, from about 1 to about 5, from about 1 to about 4, from about 1 to about 3, or from about 2 to about 3 mol % CO₂. In some aspects, the gaseous substrate may comprise less than about 20, about 15, about 10, or about 5 mol % CO₂. In another aspect, the gaseous substrate comprises no or substantially no (<about 1 mol %) CO₂.

In an aspect, the at least one acetogenic carboxydotrophic bacterium does not comprise sequence

(SEQ ID NO: 3)
taaatacttcaccctcccatttgaatattataattgatttcccccttaaa
atattagaaggaataagatattagctctaaaccaaagatttgagcctcta
tacttaagaaataattttaaaacatataacacatttaatttaaaattatt
ttaattaccttatttaaaaattaaaatataaacttagtcctatttaatta
gggactaattattaacattaaatattatattatttttatataactatatg
attaattttttaagattatattcaaaaatatgttaacagaaatta.

In an aspect, the at least one acetogenic carboxydotrophic bacterium does not comprise a sequence that is about 90% or more, about 91% or more, about 92% or more, about 93% or more, about 94% or more, about 95% or more, about 96% or more, about 97% or more, about 98% or more, about 99% or more, or about 100% identity to SEQ ID NO: 3.

In an aspect, the at least one acetogenic carboxydotrophic comprises an upstream region sequence for a hydrogenase gene cluster that comprises at least about 95%, about 96%, about 97%, about 98%, about 99% or about 100% identity to

SEQ ID NO: 2
(taatttctgctaatacatttttaaataaggtaattaaaataattttaaa
ttaaatatgttatatgttttaaaattatttcttaagcatagaggctcaaa
tctttgatttagagctaatatcttattccttctaatattttaagggggaa
atcaattataatattcaaatgggagggtgaagtattta).

In an aspect, the at least one acetogenic carboxydotrophic comprises an hydrogenase gene cluster comprising at least about 90% or more, about 91% or more, about 92% or more, about 93% or more, about 94% or more, about 95% or more, about 96% or more, about 97% or more, about 98% or more, about 99% or more, or about 100% sequence identity to SEQ ID NO: 1. In an aspect, the at least one acetogenic carboxydotrophic comprises an hydrogenase gene cluster comprising at least about 91% or more, about 92% or more, about 93% or more, about 94% or more, about 95% or more, about 96% or more, about 97% or more, about 98% or more, about 99% or more, or about 100% sequence identity to any one or more of SEQ ID NOs: 4-9. In an aspect, the hydrogenase gene cluster comprises genes fdhH, moeA, moeB, fdhD, nuoE, nuoF, nuoG, hydN, and/or hydrogenase-1. In another aspect, the hydrogenase gene cluster comprises genes nuoE, nuoF, nuoG, hydN, and/or hydrogenase-1.

As used herein, a sequence is upstream of another sequence when it is position 5′ to another sequence. A sequence is considered downstream of another sequence when it is postioned 3′ to another sequence.

Identity (e.g., percent homology) can be determined using any homology comparison software, including for example, the BlastN software of the National Center of Biotechnology Information (NCBI). According to an aspect of the invention, the identity is a global identity, i.e., an identity over the entire amino acid or nucleic acid sequences of an aspect of the invention and not over portions thereof. For example, when comparing SEQ ID NO: 2 and SEQ ID NO: 3, using a percent identity over the entire sequences provides an accurate view of the differences between the two sequences (i.e., SEQ ID NO: 2 is missing a sequence that is part of SEQ ID NO: 3).

In an aspect, the hydrogenase gene cluster in the at least one acetogenic carboxydotrophic bacterium is expressed at least about 1.5, at least about 1.75, at least about 2, at least about 2.25, at least about 2.5, at least about 2.75, at least about 3, at least about 3.25, at least about 3.5, at least about 3.75, at least about 4, at least about 4.25, or at least about 5 times more than the hydrogenase gene cluster in C. autoethanogenum is expressed under similar culturing conditions.

In an aspect, at least about 5%, at least about 6%, at least about 7%, at least about 8%, at least about 9%, at least about 10%, at least about 15%, at least about 20%, or at least about 25% more of the at least one oxygenated product is produced as compared to the amount of the at least one oxygenated product C. autoethanogenum produces under similar culturing conditions.

In an aspect, the at least one acetogenic carboxydotrophic bacterium is cultured in the bioreactor to produce an acetogenic carboxydotrophic bacterium culture. In an aspect, the acetogenic carboxydotrophic bacterium culture continuously produces at least one oxygenated product for more than about 24 hours, about 36 hours, about 48 hours, about 72 hours, about 96 hours, about 120 hours, about 144 hours, about 168 hours, about 192 hours, about 216 hours, about 250 hours, about 300 hours, about 400 hours, about 500 hours, about 600 hours, about 700 hours, about 800 hours, about 900 hours, about 1,000, 1,100 hours, about 1,200 hours, about 1,300 hours, about 1,400 hours, about 1,500 hours, about 1,600 hours, about 1,700 hours, about 1,800 hours, about 1,900 hours, about 2,000 hours, about 2,500 hours, or about 3,000 hours.

In an aspect, the present methods further comprise removing the bioreactor broth from the bioreactor to produce a removed broth.

In an aspect, the present methods further comprise removing the at least one oxygenated product from the removed broth to produce an oxygenated product-depleted removed broth. The oxygenated products may be separated or purified from the fermentation broth using any method or combination of methods known in the art, including, for example, fractional distillation, evaporation, pervaporation, gas stripping, phase separation, and extractive fermentation, including for example, liquid-liquid extraction. In certain aspects, target products are recovered from the fermentation broth by continuously removing a portion of the broth from the bioreactor, separating cells from the broth (e.g., centrifugation or filtration), and recovering one or more oxygenated products from the fermentation broth. Alcohols may be recovered, for example, by distillation.

In an aspect, the present methods further comprise removing cells of the acetogenic carboxydotrophic bacterium culture from the removed broth and/or the oxygenated product-depleted removed broth. Removal of the cells can be carried out in any suitable means, for example, by cyclones, filtration, or centrifugation. The cells may be intact cells, or the cells can be lysed and the removal can include the removal of the lysed cell components.

In an aspect, the present methods further comprise providing the oxygenated product-depleted removed broth to the bioreactor. Additional nutrients (e.g., vitamins and metals) may be added to the removed broth to replenish the oxygenated product-depleted removed broth before it is returned to the bioreactor.

In an aspect, at least about 1 gram, about 2 grams, about 3 grams, about 4 grams, about 5 grams, about 6 grams, about 7 grams, about 8 grams, about 9 grams, about 10 grams, about 11 grams, about 12 grams, about 13 grams, about 14 grams, about 15 grams, or about 20 grams of the at least one product are produced per liter of removed broth.

In an aspect, more than about 30%, about 35%, about 40%, about 45%, about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor. In this regard, from about 1% to about 30% of the oxygenated product-depleted removed broth is provided back to the bioreactor, for example, from about 1% to about 35%, from about 1% to about 40%, from about 1% to about 45%, from about 1% to about 50%, from about 1% to about 55%, from about 1% to about 60%, from about 1% to about 65%, from about 1% to about 70%, from about 1% to about 75%, or from about 1% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the removed broth is provided back to the bioreactor. In an alternative aspect, from about 50% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor.

An aspect of the invention further provides methods for improving bottoms recycle, the method comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂ and optionally H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the hydrogenase gene cluster comprises at least about 90% identity to SEQ ID NO: 1, the upstream region sequence comprises at least about 90% identity to SEQ ID NO: 2, and the conditions within the bioreactor create a bioreactor broth.

An aspect of the invention further provides methods for improving bottoms recycle, the method comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂ and optionally H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

An aspect of the invention further provides methods for converting CO, CO₂, and optionally H₂ to ethanol, the method comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂, and optionally H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the hydrogenase gene cluster comprises at least about 90% identity to SEQ ID NO: 1, the upstream region sequence comprises at least about 90% identity to SEQ ID NO: 2, and the conditions within the bioreactor create a bioreactor broth.

An aspect of the invention further provides methods for converting CO, CO₂, and optionally H₂ to ethanol, the method comprising: (a) providing to a bioreactor: (1) a gaseous substrate comprising CO, CO₂, and optionally H₂; (2) at least one acetogenic carboxydotrophic bacterium; and (3) a liquid nutrient medium; and (b) providing conditions within the bioreactor for the at least one acetogenic carboxydotrophic bacterium to convert CO, CO₂, and optionally H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth.

In an aspect, the upstream region sequence is operatively linked to the hydrogenase gene cluster. In another aspect, the upstream region sequence is operatively linked to at least the first gene of the hydrogenase gene cluster. In an aspect, the upstream region sequence is a promoter. In an aspect, the upstream region sequence is a promoter that is operatively linked to at least the first gene of the hydrogenase gene cluster.

As used herein, the term “promoter” refers to an untranscribed sequence located upstream (i.e., 5′) to the translation start codon of a gene (generally within about 1 to 1000 bp, preferably 1-500 bp, especially 1-100 bp) and which controls or influences the start of transcription of the gene.

“Increasing the efficiency,” “increased efficiency,” and the like include, but are not limited to, increasing the amount of oxygenated product-depleted removed broth that can be provided to the bioreactor without negatively impacting growth of the at least one acetogenic carboxydotrophic bacterium.

Typically the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In certain aspects, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

As used herein, a “bioreactor” refers to a bioreactor assembly. A bioreactor assembly is a group of one or more vessels suitable to contain aqueous broth and microorganisms for the bioconversion. The bioreactor assembly may contain associated equipment such as injectors, recycle loops, and agitators. As used herein, “providing the oxygenated product-depleted removed broth to the bioreactor” means that the oxygenated product-depleted removed broth can be added to any portion of a bioreactor assembly. For example, the oxygenated product-depleted removed broth may be added to a recycle loop, to the vessels that contain aqueous broth, or to any convenient location within the bioreactor assembly. The oxygenated product-depleted removed broth may be removed from one bioreactor assembly and later added to another bioreactor assembly.

Any suitable bioreactor assembly may be used in the processes of this invention. The bioreactor assembly can be the bioreactor assembly used for the bioconversion of syngas, or the bioreactor assembly can be separate. The bioreactor assemblies for use in the processes of this invention include, but are not limited to, column reactors; bubble columns; jet loop reactors; stirred tank reactors; fluidized bed reactors; trickle bed reactors; biofilm reactors, including, but not limited to membrane bioreactors; and static mixer reactors including, but not limited to, pipe reactors. One or more bioreactors may be used, and when two or more bioreactors are used, they may be in parallel or sequential operation. The bioreactor assembly can, but is not always required to, include heat exchangers; solids separation unit operations such as centrifuges, settling ponds and filters; gas/liquid separation unit operations; pumps; and equipment useful for monitoring and control of the bioreactor assembly.

A separate bioreactor assembly can, if desired, be integrated into the facility for the bioconversion of syngas to oxygenated organic compound. For instance, where the facility contains a distillation assembly, the distillation assembly may be used to remove at least a portion of the oxygenated product and to denature the aqueous fermentation broth.

As used herein, “biomass” refers to biological material living or recently living plants and animals and contains at least hydrogen, oxygen, and carbon. Biomass typically also contains nitrogen, phosphorus, sulfur, sodium, potassium, and trace metals. The chemical composition of biomass can vary from source to source and even within a source. Sources of biomass include, but are not limited to, harvested plants such as wood, grass clippings and yard waste, switchgrass, corn (including corn stover), hemp, sorghum, sugarcane (including bagas), and the like; and waste such as garbage and municipal solid waste. Biomass does not include fossil fuels such as coal, natural gas, and petroleum.

Fossil carbonaceous materials, or fossil fuels, include, but are not limited to, natural gas; petroleum including carbonaceous streams from the refining or other processing of petroleum including, but not limited to, petroleum coke, lignite, and coal.

As used herein, “aqueous broth,” or “aqueous fermentation broth,” refers to a liquid water phase which may contain dissolved compounds including, but not limited to hydrogen, carbon monoxide, and carbon dioxide.

Intermittently means from time to time and may be at regular or irregular time intervals.

Syngas means a gas, regardless of source, containing at least one of hydrogen and carbon monoxide and may, and usually does, contain carbon dioxide.

Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas. Syngas is typically produced by a gasifier or reformer (steam, autothermal or partial oxidation). Any of the aforementioned biomass sources are suitable for producing syngas. The syngas produced thereby will typically contain from about 10 to about 60 mole % CO, from about 10 to about 25 mole % CO₂ and from about 10 to about 75, often at least about 30, and preferably between about 35 and about 65, mole % H₂. The syngas may also contain N₂ and CH₄ as well as trace components such as H₂S and COS, NH₃ and HCN. Other sources of the gas substrate include gases generated during petroleum and petrochemical processing and from industrial processes. These gases may have substantially different compositions than typical syngas, and may be essentially pure hydrogen or essentially pure carbon monoxide. The gas substrate may be obtained directly from gasification or from petroleum and petrochemical processing or industrial processes or may be obtained by blending two or more streams. Also, the gas substrate may be treated to remove or alter the composition including, but not limited to, removing components by chemical or physical sorption, membrane separation, water-gas shift, and selective reaction.

The product oxygenated organic compounds produced in the processes of this invention will depend upon the microorganism or combination of microorganisms used for the fermentation and the conditions of the fermentation.

Any suitable microorganisms that have the claimed features may be utilized.

The aqueous broth is maintained under anaerobic fermentation conditions including a suitable temperature, for example, between about 25° C. and about 60° C., or in the range of about 30° C. to about 40° C. The conditions of fermentation, including the density of microorganisms and aqueous fermentation broth composition are preferably sufficient to achieve the sought conversion efficiency of hydrogen and carbon monoxide. The pH of the aqueous broth is acidic, for example between about 4 and about 6.5.

The rate of supply of the syngas under steady state conditions to a bioreactor is preferably such that the rate of transfer of carbon monoxide and hydrogen to the liquid phase matches the rate that carbon monoxide and hydrogen are bioconverted. The rate at which carbon monoxide and hydrogen can be consumed will be affected by the nature of the microorganism, the concentration of the microorganism in the aqueous fermentation broth and the fermentation conditions. As the rate of transfer of carbon monoxide and hydrogen to the aqueous fermentation broth is a parameter for operation, conditions affecting the rate of transfer such as interfacial surface area between the gas and liquid phases and driving forces are important. Preferably the feed gas is introduced into the bioreactor in the form of microbubbles. Often the microbubbles have diameters in the range of about 0.01 to about 0.5, or about 0.02 to about 0.3 millimeter.

In another aspect, the disclosure provides a method of preparing animal feed. As used herein, animal feed can be any suitable type of animal feed, such as, for example, aquatic culture (fish feed), poultry feed, cattle feed, hog feed, bird feed, etc. In an aspect, the removed cells are effective for use as animal feed. In a further aspect, the disclosure provides methods of preparing animal feed, the methods comprising: providing to a bioreactor: (1) a gaseous substrate comprising CO, CO₂, and optionally H₂; (2) at least one acetogenic carboxydotrophic bacterium; and (3) a liquid nutrient medium; providing conditions within the bioreactor for the at least one acetogenic carboxydotrophic bacterium to convert CO, CO₂ and H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the hydrogenase gene cluster comprises at least about 90% identity to SEQ ID NO: 1, the upstream region sequence comprises at least about 90% identity to SEQ ID NO: 2, and the conditions within the bioreactor create a bioreactor broth, removing the bioreactor broth from the bioreactor to produce a removed broth, removing the at least one oxygenated product from the removed broth to produce an oxygenated product- depleted removed broth, and removing cells of the acetogenic carboxydotrophic bacterium culture from the removed broth and/or the oxygenated product-depleted removed broth, wherein the removed cells are effective for use as animal feed. In a further aspect, the disclosure provides methods of preparing animal feed, the methods comprising: providing to a bioreactor: (1) a gaseous substrate comprising CO, CO₂, and optionally H₂; (2) at least one acetogenic carboxydotrophic bacterium; and (3) a liquid nutrient medium; providing conditions within the bioreactor for the at least one acetogenic carboxydotrophic bacterium to convert CO, CO₂ and H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth, removing the bioreactor broth from the bioreactor to produce a removed broth, removing the at least one oxygenated product from the removed broth to produce an oxygenated product-depleted removed broth, and removing cells of the acetogenic carboxydotrophic bacterium culture from the removed broth and/or the oxygenated product-depleted removed broth, wherein the removed cells are effective for use as animal feed. In some embodiments, the method of preparing animal feed is useful for producing aquatic culture containing relatively low amounts of one or more toxic metals, such as mercury, iron, nickel, etc.

In another aspect, the disclosure provides a method of preparing fertilizer. In an aspect, the removed cells are effective for use as fertilizer. In a further aspect, the disclosure provides methods of preparing fertilizer, the methods comprising: providing to a bioreactor: (1) a gaseous substrate comprising CO, CO₂, and optionally H₂; (2) at least one acetogenic carboxydotrophic bacterium; and (3) a liquid nutrient medium; providing conditions within the bioreactor for the at least one acetogenic carboxydotrophic bacterium to convert CO, CO₂ and H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the hydrogenase gene cluster comprises at least about 90% identity to SEQ ID NO: 1, the upstream region sequence comprises at least about 90% identity to SEQ ID NO: 2, and the conditions within the bioreactor create a bioreactor broth, removing the bioreactor broth from the bioreactor to produce a removed broth, removing the at least one oxygenated product from the removed broth to produce an oxygenated product-depleted removed broth, and removing cells of the acetogenic carboxydotrophic bacterium culture from the removed broth and/or the oxygenated product-depleted removed broth, wherein the removed cells are effective for use as fertilizer. In a further aspect, the disclosure provides methods of preparing fertilizer, the methods comprising: providing to a bioreactor: (1) a gaseous substrate comprising CO, CO₂, and optionally H₂; (2) at least one acetogenic carboxydotrophic bacterium; and (3) a liquid nutrient medium; providing conditions within the bioreactor for the at least one acetogenic carboxydotrophic bacterium to convert CO, CO₂ and H₂ to at least one oxygenated product; wherein the at least one acetogenic carboxydotrophic bacterium comprises an upstream region sequence for a hydrogenase gene cluster, the hydrogenase gene cluster comprises at least a first gene, the upstream region sequence is upstream of the first gene, the upstream region sequence comprises at least about 90% identity to SEQ ID NO: 2, and the conditions within the bioreactor create a bioreactor broth, removing the bioreactor broth from the bioreactor to produce a removed broth, removing the at least one oxygenated product from the removed broth to produce an oxygenated product-depleted removed broth, and removing cells of the acetogenic carboxydotrophic bacterium culture from the removed broth and/or the oxygenated product-depleted removed broth, wherein the removed cells are effective for use as fertilizer.

In an aspect, the removed cells are effective for landfill application or land application as fertilizer or animal feed. In an aspect, the removed cells useful for fertilizer, animal feed, landfill application, and/or land application contain protein, fat, carbohydrate, and/or minerals, e.g., 86% protein, 2% fat, 2% minerals, 10% carbohydrate. The removed cells can be effective for landfill application (e.g., to Class A solids), land application as fertilizer, or feed for animals, such as aquatic culture (fish feed), poultry feed, cattle feed, hog feed, etc. In the case of fish feed, advantageously, in some embodiments, the fish feed contains less total toxic metals as compared with conventional fish meal.

In some aspects, the removed cells contain about 25 to about 50 wt. % solids, such as about 25 to about 40 wt. % solids. The amount of recovered solids is advantageous because it contains protein, carbohydrates, minerals, and potentially vitamins of nutritional value for plants and animals.

The removed cells can be used wet or dry. For example, in some aspects, the removed cells are effective for use as a wet fertilizer. In some aspects, the method further comprises drying the removed cells to form a “dried cake”, and the resulting dried cake is effective for use as dry fertilizer, animal feed or fish feed, or any combination thereof. The drying of the removed cells can also be used as a means to concentrate the removed cells. However, the removed cells can be dried using any suitable means.

The respective compositions of the animal feed and fertilizer are generally similar because they are mainly composed of microbial proteins and/or carbohydrates. In some embodiments, the animal feed and/or fertilizer contains protein (e.g. from about 30 wt. % to about 90 wt. %, such as from about 60 wt. % to about 90 wt. %), fat (e.g. from about 1 wt. % to about 12 wt. %, such as from about 1 wt. % to about 3 wt. %), carbohydrate (e.g. from about 5 wt. % to about 60 wt. %, such as from about 15 wt. % to about 60 wt. %, or from about 5 wt. % to about 15 wt. %) and/or minerals such as sodium, potassium, copper etc. (e.g. from about 1 wt. % to about 20 wt. %, such as from about 1 wt. % to about 3 wt. %). For example, the animal feed and/or fertilizer can contain about 86% protein, about 2% fat, about 2% minerals, and about 10% carbohydrate.

### Examples of Non-Limiting Aspects of the Disclosure

Aspects, including embodiments, of the present subject matter described herein may be beneficial alone or in combination, with one or more other aspects or aspects. Without limiting the foregoing description, certain non-limiting aspects of the disclosure numbered 1-18 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A method for efficient fermentation broth recycle, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂ and optionally H₂ to at least one oxygenated product,
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth.

(2) The method of aspect (1), wherein the oxygenated product is ethanol.

(3) The method of aspect (1), wherein the at least one acetogenic carboxydotrophic bacterium does not comprise SEQ ID NO:3.

(4) The method of aspect (1), wherein the at least one acetogenic carboxydotrophic bacterium comprises an upstream region sequence for a hydrogenase gene cluster that comprises at least about 95% identity to SEQ ID NO: 2.

(5) The method of aspect (1), wherein the hydrogenase gene cluster comprises at least about 90% sequence identity to SEQ ID NO: 1.

(6) The method of aspect (1), wherein the hydrogenase gene cluster in the at least one acetogenic carboxydotrophic bacterium is expressed at least about 1.5 times more than the hydrogenase gene cluster in Clostridium autoethanogemum is expressed under similar culturing conditions.

(7) The method of aspect (1), wherein at least about 5% more of the at least one oxygenated product is produced as compared to the amount of the at least one product Clostridium autoethanogemm produces under similar culturing conditions.

(8) The method of aspect (1), wherein the at least one acetogenic carboxydotrophic bacterium is cultured in the bioreactor to produce an acetogenic carboxydotrophic bacterium culture.

(9) The method of aspect (8), wherein the acetogenic carboxydotrophic bacterium culture continuously produces at least one oxygenated product for more than about 24 hours.

(10) The method of aspect (8), further comprising:
(c) removing the bioreactor broth from the bioreactor to produce a removed broth;
(d) removing the at least one oxygenated product from the removed broth to produce an oxygenated product-depleted removed broth;
(e) removing cells of the acetogenic carboxydotrophic bacterium culture from the removed broth and/or the oxygenated product-depleted removed broth; and
(f) providing the oxygenated product-depleted removed broth to the bioreactor.

(11) The method of aspect (10), wherein at least about 1 gram of the at least one oxygenated product is produced per liter of removed broth.

(12) The method of aspect (10), wherein more than 80% of the oxygenated product-depleted removed broth is provided to the bioreactor.

(13) The method of any one of claims 10-12, wherein the removed cells are effective for use as animal feed.

(14) The method of any one of claims 10-13, wherein the removed cells are effective for use as fertilizer.

(15) A method for improving bottoms recycle, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂, and optionally H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth.

(16) A method for converting CO, CO₂, and optionally H₂ to ethanol, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂, and optionally H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth.

(17) A method of preparing animal feed, the method comprising:
(a) providing to a bioreactor: (1) a gaseous substrate comprising CO, CO₂, and optionally H₂; (2) at least one acetogenic carboxydotrophic bacterium; and (3) a liquid nutrient medium;
(b) providing conditions within the bioreactor for the at least one acetogenic carboxydotrophic bacterium to convert CO, CO₂ and H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth;
(c) removing the bioreactor broth from the bioreactor to produce a removed broth;
(d) removing the at least one oxygenated product from the removed broth to produce an oxygenated product-depleted removed broth; and
(e) removing cells of the acetogenic carboxydotrophic bacterium culture from the removed broth and/or the oxygenated product-depleted removed broth;
wherein the removed cells are effective for use as animal feed.

(18) A method of preparing fertilizer, the method comprising:
(a) providing to a bioreactor: (1) a gaseous substrate comprising CO, CO₂, and optionally H₂; (2) at least one acetogenic carboxydotrophic bacterium; and (3) a liquid nutrient medium;
(b) providing conditions within the bioreactor for the at least one acetogenic carboxydotrophic bacterium to convert CO, CO₂ and H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth;
(c) removing the bioreactor broth from the bioreactor to produce a removed broth;
(d) removing the at least one oxygenated product from the removed broth to produce an oxygenated product-depleted removed broth; and
(e) removing cells of the acetogenic carboxydotrophic bacterium culture from the removed broth and/or the oxygenated product-depleted removed broth;
wherein the removed cells are effective for use as fertilizer.

The following examples further illustrate the invention but, of course, should not be construed as in any way limiting its scope.

### Example 1

This example demonstrates that the amount of gene expression in the hydrogenase gene cluster of SB1 is different than that of C. autoethanogemim.

SB1 and C. autoethanogemim were cultured under identical steady state conditions and then analyzed using RNA seq analysis to determine the level of expression of nine genes. The genes (protein, function) are as follows: fdhH (formate dehydrogenase H, oxidoreductase), moeA (molybdopterin molybdenumtransferase), moeB (molybdopterin-guanine dinucleotide biosynthesis adapter protein), fdhD (Formate dehydrogenase accessory protein FdhD), nuoE (NADH-quinone oxidoreductase subunit E), nuoF (NADH-quinone oxidoreductase subunit F), nuoG (NADH-quinone oxidoreductase subunit G), hydN (Electron transport protein hydN), and hydrogenase-1 (hydrogenase).

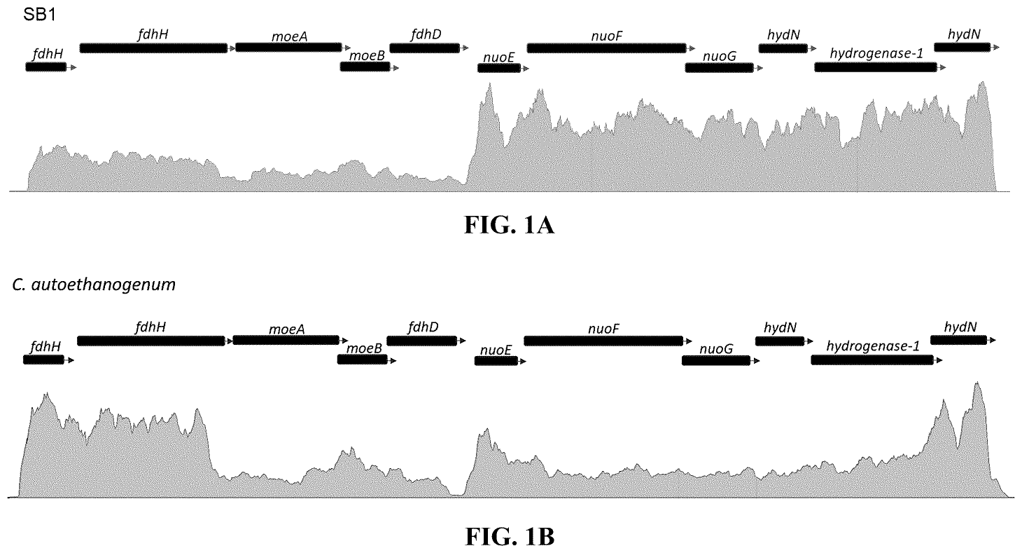

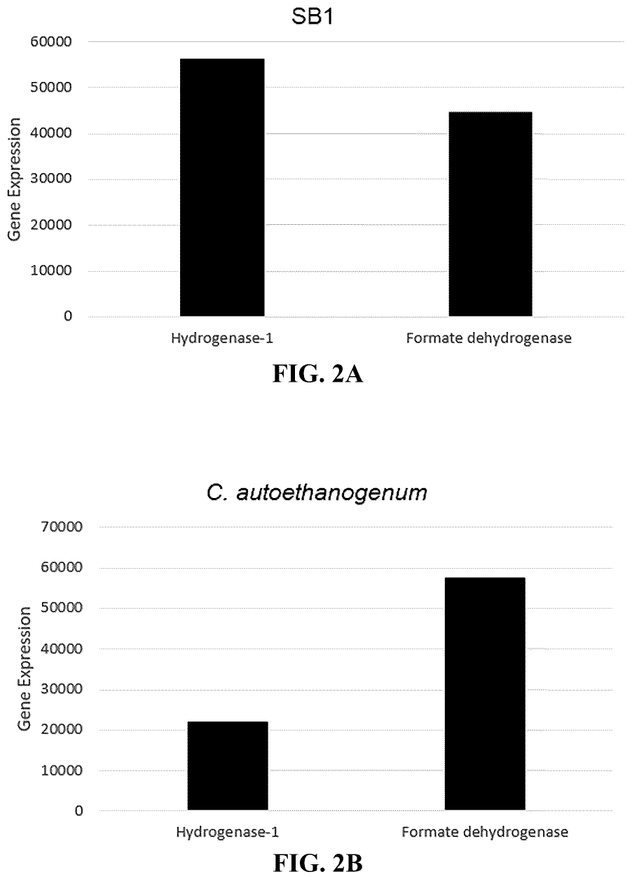

The read density derived from RNA seq data (which is proportional to the level of gene expression) for each organism were plotted. As seen in FIGS. 1A and 1B, the read density levels differ between SB1 and C. autoethanogenum. Higher values on the plots represent higher levels of gene expression. FIGS. 2A and 2B are graphs showing the average amount of gene expression for hydrogenase-1 gene cluster and formate dehydrogenase gene cluster in organisms SB1 (FIG. 2A) and C. autoethanogemim (FIG. 2B). Formate dehydrogenase represents the averaged gene expression of the genes involved in formate dehydrogenase (fdhH-fdhD) and hydrogenase-1 represents the averaged gene expression of the genes involved in hydrogenase (nuoE-hydN). FIG. 2A is a plot of the data derived from analysis of data used to generate FIG. 1A and FIG. 2B is the plot of the data derived from analysis of the data used to generate FIG. 1B. In SB1, the averaged gene expression for hydrogenase-1 gene cluster is higher than averaged gene expression for formate dehydrogenase gene cluster. In C. autoethanogemim, the averaged gene expression is lower for hydrogenase-1 than for formate dehydrogenase gene cluster.

### Example 2

This example demonstrates that SB1 tolerates high levels of bottoms recycle.

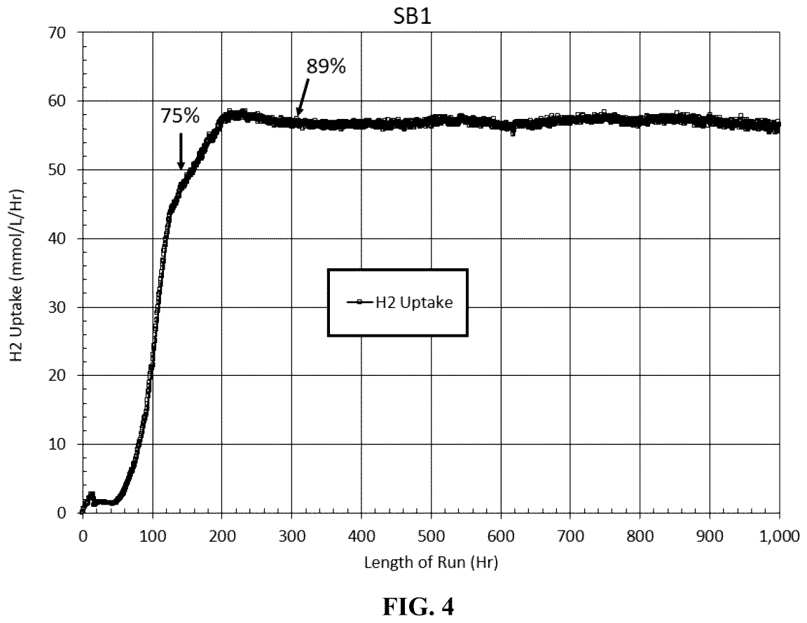

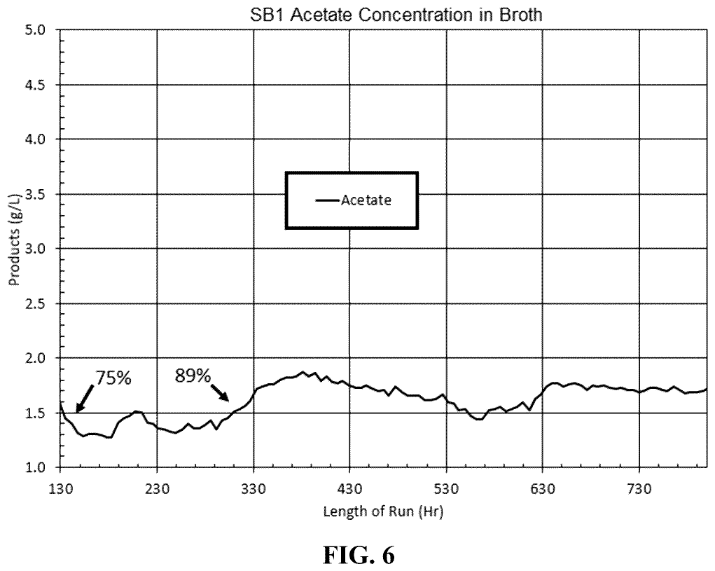

SB1 was placed in a fermentation vessel in the presence of syngas and the amount of hydrogen update (mmol/L/Hr) was recorded. As seen in FIG. 4, at 143 hours (arrow), the cell media was switched to 75% bottoms recycle. At 307 hours (arrow), the cell media was switched to 89% bottoms recycle. The hydrogen uptake of SB1 was steady for at least the next 700 hours. As seen in FIG. 6, acetate concentration was steady for at least the next 500 hours.

The data shows that increasing bottoms recycle did not negatively impact the growth or productivity of SB1 and that SB1 can tolerate the bottoms recycle of at least 89% without negative impacts.

### Comparative Example 3

This example demonstrates that C. autoethanogemm does not tolerate 85% bottoms recycle.

C. autoethanogemum was placed in a fermentation vessel in the presence of syngas. At 558 hours, the cell media was switched to 85% bottoms recycle.

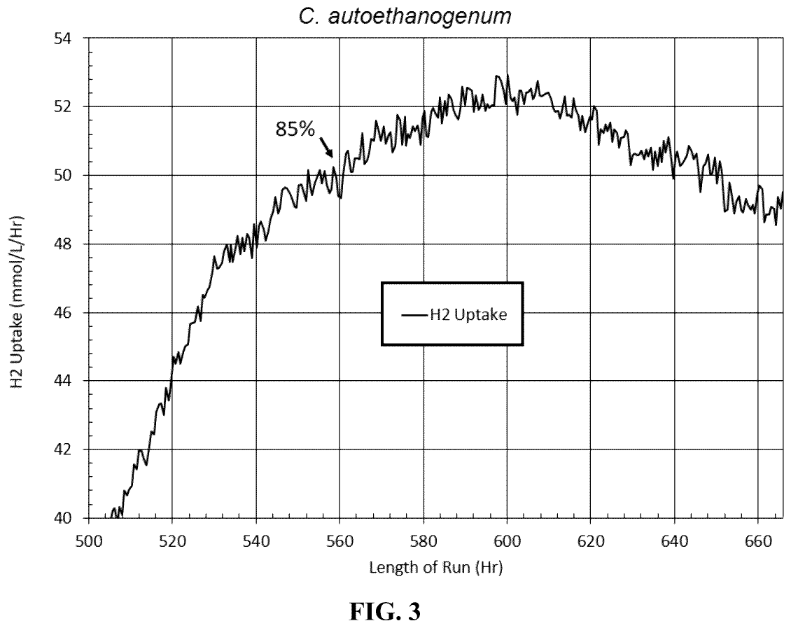

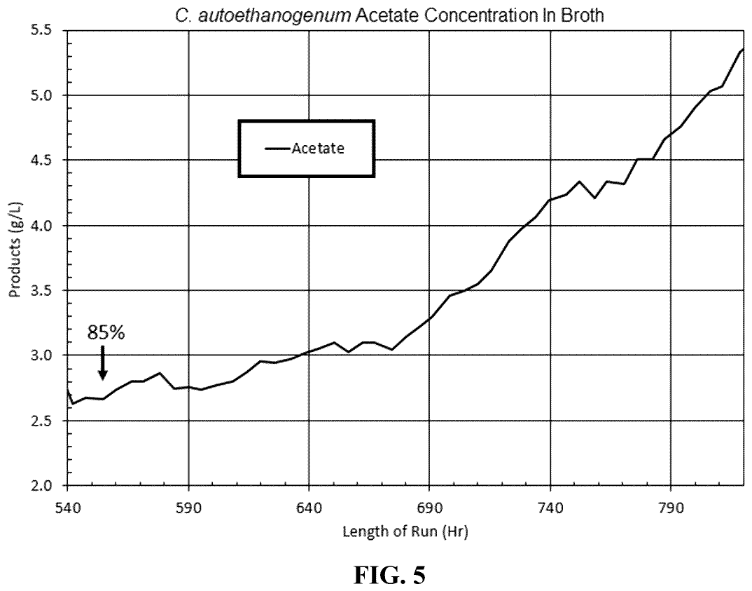

As seen in FIG. 3, within 100 hours of the switch to 85% bottoms recycle, the hydrogen uptake (mmol/L/hr) began to decline. As seen in FIG. 5, the acetate level began to increase. These data indicate a reduction in the growth and productivity of C. autoethanogemum and poor tolerance of the 85% bottoms recycle.

### Example 4

SB1 and C. autoethanogemim (strain DSM 10061) were cultured under identical steady state conditions and then analyzed.

Direct expression comparisons between C. autoethanogemum and SB1 were carried out by Igenbio, Inc. (Chicago, IL) using a Pan-genome approach (see Medini, et al., Current Opinion in Genetics & Development, 15(6):589-594 (2005)). The ERGO™ genome analysis and discovery system (see Overbeek et al., Nucleic Acids Research, 31(1): 164-171 (2003)) was used to identify bidirectional best hits between C. autoethanogemum and SB1, and generate a core genome. RNA-Seq data from C. autoethanogenum and SB1 were used for analysis with the core genome containing the bi-directional best hits.

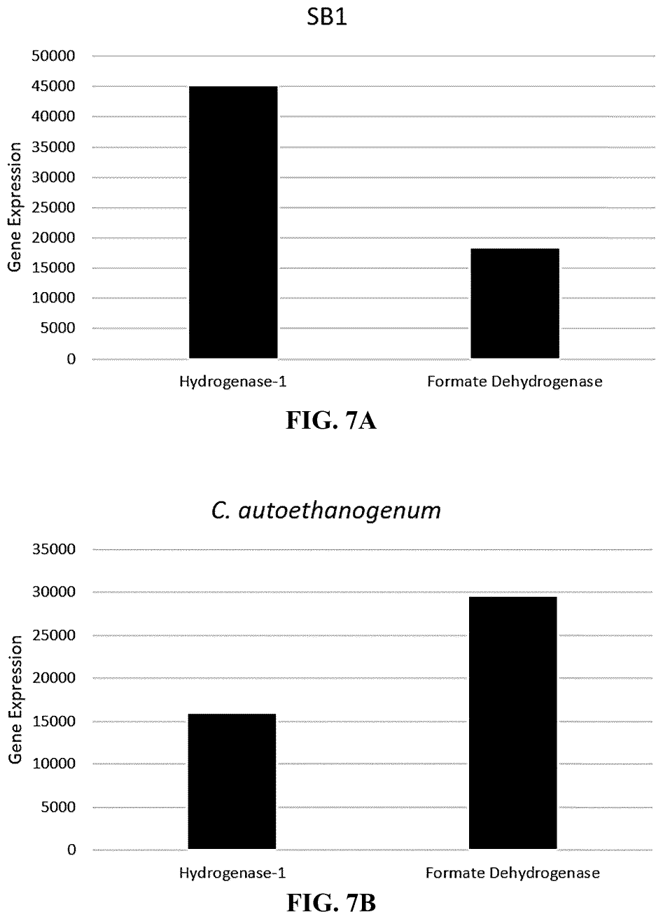

FIGS. 7A and 7B are bar graphs showing the averaged gene expression for the formate dehydrogenase and hydrogenase genes in SB1 (FIG. 7A) and C. autoethanogemum (FIG. 7B). Gene expression for genes in formate dehydrogenase (fdhH-fdhD) and hydrogenase gene clusters (nuoE-hydN) was then averaged and plotted. In SB1, the averaged gene expression for the hydrogenase gene cluster is higher than the averaged gene expression for formate dehydrogenase gene cluster. In C. autoethanogenum, the averaged gene expression is lower for hydrogenase than for the formate dehydrogenase gene cluster. In addition, hydrogenase expression is higher in SB1 than in C. autoethanogemum, whereas the opposite is true for formate dehydrogenase expression.

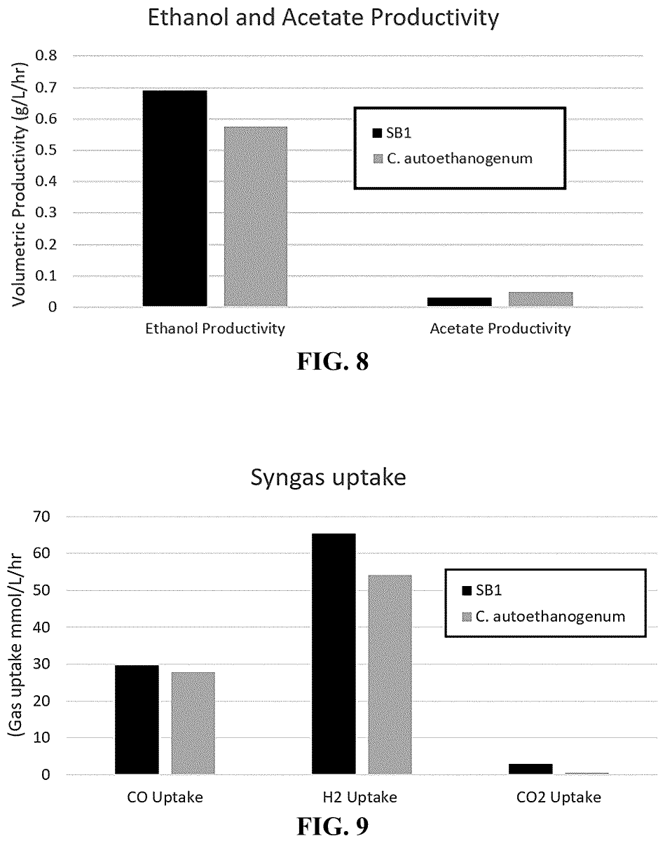

FIG. 8 shows the ethanol and acetate volumetric productivity in SB1 and C. autoethanogenum. Values were derived from broth ethanol and acetate concentrations in fermentor broth (measured by gas chromatography). FIG. 9 shows the rates of CO, hydrogen, and CO₂ uptake in SB1 and C. autoethanogemum. Gas uptakes were measured by process mass spectrometry. CO uptake is largely identical for the two organisms, whereas hydrogen uptake is higher for SB1. CO₂ uptake is also higher for SB1 as compared to C. autoethanogemim.

### Example 5

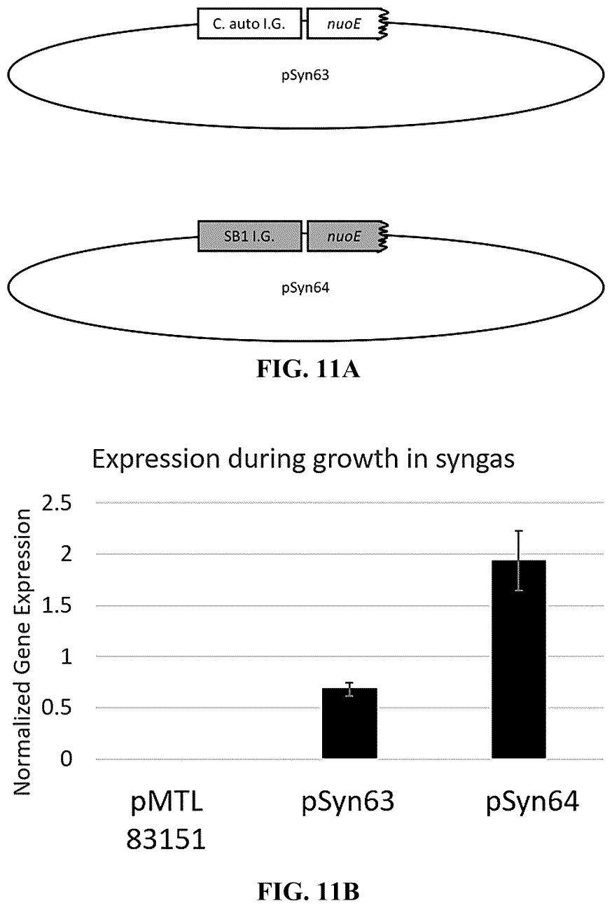

This example demonstrates that the intergenic region from SB1 is sufficient for elevated expression of hydrogenase. Fragments corresponding to the intergenic (I.G) (upstream) region and initial 100 bp of nuoE from C. autoethanogemim (C. auto) and SB1 were amplified via PCR. nuoE is the first gene of the cluster encoding the primary bifurcating hydrogenase, and the initial 100 bp of the nuoE genes from both strains are identical in sequence. The intergenic-nuoE regions from C. autoethanogemim and SB1 strains were then cloned into plasmid pMTL83151 to form plasmids pSyn63 and pSyn64, respectively (FIG. 11A). The plasmids were then transformed into strain SB1 and selected for using 2 ug/ml thiamphenicol. Plasmid pMTL83151 was also transformed as a negative control.

Cells containing pSyn63, pSyn64, and pMTL83151 were cultured in fructose media (Mock, et al., J. Bacteriol., 197(18): 2965-2980 (2015)). The cells were washed and then cultured in the same medium in the presence of syngas as a carbon and energy source. Cells were then captured by freezing in liquid nitrogen, RNA extracted, converted to cDNA, and nuoE expression quantified using plasmid specific qPCR (FIG. 11B). Expression of catP, encoding thiamphenicol resistance was also quantified in the same manner and used to normalize expression. FIGS. 11A and 11B taken together show that the intergenic region of SB1 is sufficient for higher expression of hydrogenase genes.

These results show that the intergenic region from SB1 is sufficient to produce higher levels of nuoE expression than the intergenic region from C. autoethanogemum, indicating that differences in gene expression originate from this intergenic region.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for efficient fermentation broth recycle, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂, and optionally H₂ to at least one oxygenated product,
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity to SEQ ID NO: 2, and the conditions within the bioreactor create a bioreactor broth.

2. The method of claim 1, wherein the oxygenated product is ethanol.

3. The method of claim 1, wherein the at least one acetogenic carboxydotrophic bacterium does not comprise SEQ ID NO: 3.

4. The method of claim 1, wherein the at least one acetogenic carboxydotrophic bacterium comprises an upstream region sequence for a hydrogenase gene cluster that comprises at least 95% identity to SEQ ID NO: 2.

5. The method of claim 1, wherein the hydrogenase gene cluster comprises at least about 90% sequence identity to SEQ ID NO: 1.

6. The method of claim 1, wherein the hydrogenase gene cluster in the at least one acetogenic carboxydotrophic bacterium is expressed at least about 1.5 times more than the hydrogenase gene cluster in Clostridium autoethanogenum is expressed under similar culturing conditions.

7. The method of claim 1, wherein at least about 5% more of the at least one oxygenated product is produced as compared to the amount of the at least one oxygenated product Clostridium autoethanogenum produces under similar culturing conditions.

8. The method of claim 1, wherein the at least one acetogenic carboxydotrophic bacterium is cultured in the bioreactor to produce an acetogenic carboxydotrophic bacterium culture.

9. The method of claim 8, wherein the acetogenic carboxydotrophic bacterium culture continuously produces at least one oxygenated product for more than about 24 hours.

10. The method of claim 8, further comprising:
(c) removing the bioreactor broth from the bioreactor to produce a removed broth;
(d) removing the at least one oxygenated product from the removed broth to produce an oxygenated product-depleted removed broth;
(e) removing cells of the acetogenic carboxydotrophic bacterium culture from the removed broth and/or the oxygenated product-depleted removed broth; and
(f) providing the oxygenated product-depleted removed broth to the bioreactor.

11. The method of claim 10, wherein at least about 1 gram of the at least one oxygenated product is produced per liter of removed broth.

12. The method of claim 10, wherein more than about 80% of the oxygenated product-depleted removed broth is provided to the bioreactor.

13. The method of claim 10, wherein the removed cells are effective for use as animal feed.

14. The method of claim 10, wherein the removed cells are effective for use as fertilizer.

15. A method for improving bottoms recycle, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂ and H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity SEQ ID NO: 2, and the conditions within the bioreactor create a bioreactor broth.

16. A method for converting CO, CO₂, and optionally H₂ to ethanol, the method comprising:
(a) providing to a bioreactor: (1) a gaseous substrate comprising CO, CO₂, and optionally H₂; (2) at least one acetogenic carboxydotrophic bacterium; and (3) a liquid nutrient medium; and
(b) providing conditions within the bioreactor for the at least one acetogenic carboxydotrophic bacterium to convert CO, CO₂, and optionally H₂ to at least one oxygenated product;
wherein the at least one acetogenic carboxydotrophic bacterium comprises an upstream region sequence for a hydrogenase gene cluster,
the hydrogenase gene cluster comprises at least a first gene,
the upstream region sequence is upstream of the first gene,
the upstream region sequence comprises at least about 90% identity SEQ ID NO: 2, and the conditions within the bioreactor create a bioreactor broth.

17.-18. (canceled)
